(19) United States
(12) Patent Application Publication
(10) Pub. No.: US 2016/0339103 A1
(43) Pub. Date: Nov. 24, 2016

(54) Functional Micelles for Hard Tissue Targeted Delivery of Chemicals

(71) Applicant: Board of Regents of the University of Nebraskia, Lincoln, NE (US)

(72) Inventors: Dong Wang, Omaha, NE (US); Xue Li, Omaha, NE (US); Xin-Ming Liu, Omaha, NE (US); Richard Reinhardt, Roca, NE (US)

(21) Appl. No.: 15/227,246

(22) Filed: Aug. 3, 2016

Related U.S. Application Data
(63) Continuation of application No. 13/002,538, filed on Mar. 18, 2011, now abandoned.
(60) Provisional application No. 61/134,343, filed on Jul. 9, 2008.
(60) Provisional application No. 61/207,132, filed on Feb. 9, 2009.

(51) Int. Cl. A61K 47/10; A61K 47/24; A61K 31/366; A61K 8/34; A61K 8/90; A61Q 11/00; A61K 8/49; A61K 8/02; A61K 9/107; A61K 9/00; C08L 71/02; A61K 31/045; A61K 8/55

(52) U.S. Cl. CPC A61K 47/10; A61K 31/045; A61K 47/24; A61K 31/366; A61K 8/34; A61K 8/90; A61K 8/55; A61K 8/498; A61K 8/0291; A61K 9/1075; A61K 9/0063; C08L 71/02; A61Q 11/00; A61K 2800/92; A61K 2800/654; A61K 2800/652; A61K 2800/56; A61K 2800/54

(57) ABSTRACT

Compositions and methods for targeting agents to hard tissue are provided.

This application is a continuation application of U.S. patent application Ser. No. 13/002,538, filed Mar. 18, 2011, which is a §371 application of PCT/US2009/050140, filed Jul. 9, 2009, which claims priority under 35 U.S.C. §119(e) to U.S. Provisional Patent Application No. 61/134,343, filed on Jul. 9, 2008 and to U.S. Provisional Patent Application No. 61/207,132, filed on Feb. 9, 2009. The foregoing applications are incorporated by reference herein.

This invention was made with government support under AR053325 awarded by the National Institutes of Health. The government has certain rights in the invention.

### FIELD OF THE INVENTION

The present invention relates to carriers of chemicals (e.g., drugs) and methods of use thereof. More specifically, the instant invention relates to hard tissue (e.g., bone and tooth) targeting micelles.

### BACKGROUND OF THE INVENTION

Hard tissues, including tooth and bone are hosts to a wide variety of diseases, such as dental caries, osteoporosis and bone cancer, etc. Many therapeutic agents have been developed. However, their success has been largely limited by the fact that most of them do not have any hard tissue specificity and could not maintain the effective concentration at the hard tissue disease sites.

For example, dental caries is defined as the localized destruction of susceptible dental hard tissues by acidic by-products from bacterial fermentation of dietary carbohydrates (Selwitz et al. (2007) Lancet 369:51-9). Overpopulation of the oral cavity by acid-producing bacteria is one of the three main pathological factors highlighted in the cariogenic process (Featherstone et al. (2003) J. Calif. Dent. Assoc., 31:257-69). To control or even eradicate dental caries, one must focus on the bacterial aspect of the disease (Featherstone, J. D. (2000) J. Am. Dent. Assoc., 131:887-99).

Successful antimicrobial therapy against cariogenic bacteria largely depends on two major factors at the tissue level: the specificity of the antimicrobial and the maintenance of its effective local concentration. For example, chlorhexidine digluconate has been shown to be effective in reducing the levels of Streptococcus Mutans, but not Lactobacilli, in the human dental plaque, which may be partially attributed to its enamel binding capability (Anderson, M. H. (2003) J. Calif. Dent. Assoc., 31:211-4). Many available antimicrobials are active against cariogenic bacteria. However, most of them are not retained on tooth surfaces upon exposure. Therefore, the development of a retention mechanism that would maintain the antimicrobial local concentration on tooth surfaces is needed to deliver an effective therapy (Liu et al. (2006) J. Drug Target, 14:583-97; Featherstone, J. D. (2006) BMC Oral Health 6(Suppl 1):S8).

Various delivery systems have been developed to maintain drug concentration in the oral cavity. These include bioadhesive tablets (Ali et al. (2002) Int. J. Pharm., 238:93-103; Giunchedi et al. (2002) Eur. J. Pharm. Biopharm., 53:233-9; Minghetti et al. (1997) Boll. Chim. Farm., 136:543-8), bioadhesive patches/films (Nafee et al. (2003) Acta Pharm., 53:199-212; Senel et al. (2000) Int. J. Pharm. 193:197-203), and bioadhesive gels and semisolids (Jones (1999) J. Pharm. Sci., 88:592-8; Schiff, T. (2007) J. Clin. Dent., 18:79-81; Vinholis et al. (2001) Braz. Dent. J., 12:209-13). Their mechanism of retention is based upon the bioadhesive polymers, which would adhere to the mucosal layer of the oral cavity. Though generally effective in maintaining drug presence in the oral cavity, these formulations provide the highest drug concentration at the mucosal epithelia instead of teeth surface. Local irritation at the site of adhesion and the uncomfortable sensation of a foreign object often lead to poor patient compliance (Mulhbacher et al. (2006) Int. J. Biol. Macromol., 40:9-14; Sudhakar et al. (2006) J. Control Release, 114:15-40). To bring direct and long lasting interaction of antimicrobials with teeth, varnish formulations have also been developed. They are generally applied by dental health practitioners during routine office visit. The long-term benefit of the periodic treatment, however, is limited due to the episodic nature dental caries.

### SUMMARY OF THE INVENTION

In accordance with the instant invention, methods for treating, inhibiting, and or preventing an oral disease or disorder in a subject are provided. In a particular embodiment, the methods comprise administering to a subject a composition comprising: a) micelles comprising i) at least one amphiphilic block copolymer linked to at least one tooth targeting moiety and ii) at least one compound (e.g., a biologically active agent); and, optionally, b) at least one pharmaceutically acceptable carrier. In a particular embodiment, the oral disease or disorder is dental caries. In yet another embodiment, the compound is an antimicrobial agent such as farnesol. In yet another embodiment, the tooth targeting moiety is alendronate.

According to another aspect of the instant invention, methods for treating, inhibiting, and/or preventing a bone disease or disorder in a subject are provided. In a particular embodiment, the methods comprise administering to a subject a composition comprising: a) micelles comprising i) at least one amphiphilic block copolymer linked to at least one bone targeting moiety and ii) at least one compound (e.g., a bone related therapeutic agent); and, optionally, b) at least one pharmaceutically acceptable carrier. In a particular embodiment, the bone related therapeutic agent is a chemotherapeutic agent. In yet another embodiment, the bone disease or disorder is bone cancer. In still another embodiment, the bone targeting moiety is alendronate.

In accordance with another aspect of the instant invention, compositions for performing the methods of the instant invention are provided. In a particular embodiment, the compositions comprise: a) micelles comprising i) at least one amphiphilic block copolymer linked to at least one tooth or bone targeting moiety and ii) at least one compound (e.g., a biologically active agent); and, optionally, b) at least one pharmaceutically acceptable carrier. In a particular embodiment of the instant invention, the compositions may be selected from the group consisting of a mouthwash, toothpaste, dentifrice, film, dental floss coating, tooth powder, topical oral gel, mouth rinse, denture product, mouth spray, lozenge, oral tablet, chewable tablet, and chewing gum.

### BRIEF DESCRIPTIONS OF THE DRAWING

FIG. 1 provides a schematic for the synthesis of Pluronic® 123-alendronate conjugate (ALN-P123).

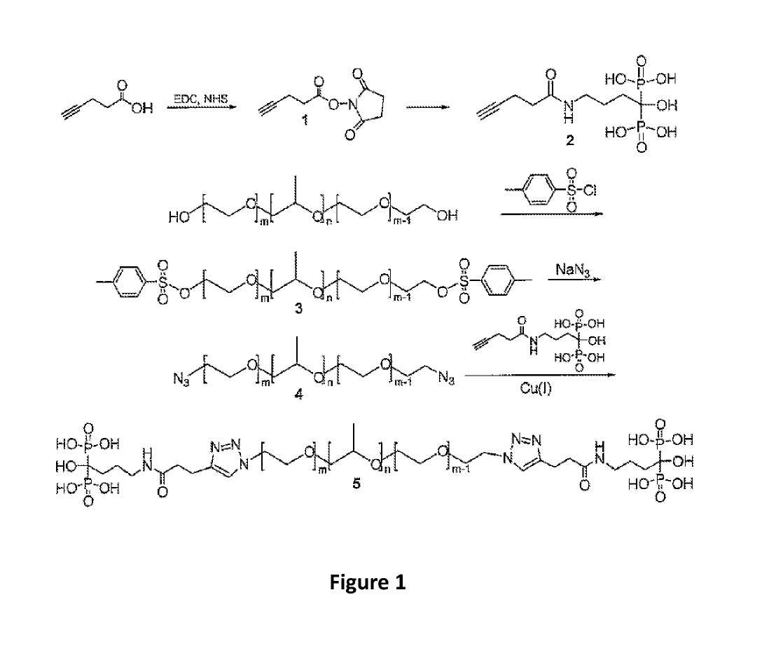

FIG. 2A is a graph demonstrating the binding kinetics of tooth binding micelles containing different amount of ALN-P123 (ALN-P123: total polymer, w/w) to hydroxyapatite (HA) surface. FIG. 2B provides a graph showing the in vitro release of tooth-binding micelles loaded with different amount of farnesol (farnesol: polymer, w/w) on HA surface.

FIG. 3 is a graph of the average number of colony forming units (cfu) of S. mutans recovered per hydroxyapatite disc after 48 hours incubation. Bars A, B, C, and D are tooth binding micelle solutions containing 1.6% P85 and 0.4% ALN-P123 encapsulating 0.4%, 0.7%, 1% and 0% farnesol, respectively. Bar E is a non-binding micelle solution containing 2% P85 encapsulating 1% farnesol. Bar F is an ethanol solution containing 1% farnesol. Bar G is a blank control. All percentages are in weight.

FIG. 4A is a graph showing the binding ratio of Rhodamine B (RB), RB labeled P123 micelles and ALN-P123 micelles to hydroxyapatite after 30 minute incubation. FIG. 4B is a graph of the binding kinetics of RB labeled bone-targeting micelles.

FIG. 5 is a graph of the in vitro release of bone-targeting micelles and non-targeting micelles on HA surface.

FIG. 6 is a graph of the in vivo anabolic effect of bone targeting micelles in mice. TMS: simvastatin loaded bone-targeting micelles, TME: empty bone-targeting micelles, NMS: simvastatin loaded non-targeting micelles, oral: simvastatin solution, and control: not treated.

### DETAILED DESCRIPTION OF THE INVENTION

To address the shortcomings and problems associated with previous delivery systems, a simple hard tissue (e.g., bone and tooth) targeting micellar delivery platform is provided herein that effectively maintains drug concentration on the applied hard tissue surface. By covalently attaching biomineral-binding moieties (e.g., bisphosphonate, acidic peptides) to the chain termini of biocompatible block copolymers (e.g., Pluronics®, block copolymers composed of poly(ethylene glycol) (PEG) and poly(D,L-lactic acid) (PLA) (e.g., PEG-PLA-PEG)), the formed micelles are capable of binding to the surface of hard tissue immediately upon exposure. The immobilized micelles then act as a drug reservoir and release the encapsulated chemicals (e.g., therapeutic agents or fragrants) gradually. In contrast to previously developed formulations, the tooth-binding micelles of the instant invention can be formulated into mouth rinse and other orally acceptable aqueous solutions. As such, the instant invention has the benefit of simple application, cultural acceptance, and improved patient compliance.

Maintenance of the effective local concentration of antimicrobials at the tooth surface is critical for management of cariogenic bacteria (e.g., S. Mutans) in the oral cavity. Indeed, the antimicrobial either prevents (e.g., inhibits) biofilm formation and subsequent dental caries manifestation or treats (e.g., reduces) existing biofilms. Herein, the design of a simple tooth-binding micellar drug delivery platform is provided that effectively binds to the tooth surface. To achieve tooth targeting, the chain termini of biocompatible copolymers (e.g., Pluronic®, PEG-PLA-PEG, etc.) can be modified with a biomineral-binding moiety (e.g, alendronate, acidic peptides, etc.). Micelles formulated with this polymer are shown herein to be able to swiftly bind to hydroxyapatite (HA) which is a model of the tooth surface as the main component of tooth enamel. The micelles also gradually release the encapsulated model antimicrobial (e.g., farnesol). These tooth-binding micelles are typically negatively charged and have an average effective hydrodynamic diameter (D_{eff}) of less than 100 nm. In a preliminary in vitro biofilm inhibition study, the farnesol-containing tooth-binding micelles were found to be able to provide significantly stronger inhibition of S. Mutans UA159 mediated biofilm formation compared to the control groups (e.g., farnesol, non-binding farnesol micelles, empty teeth-binding micelles, and no treatment).

Antimicrobials are typically hydrophobic. Indeed, farnesol is a hydrophobic compound with a water solubility of only 1.2 mM (Hornby et al. (2001) Appl. Environ. Microbiol., 67:2982-92). In previous investigations, organic solvents (e.g., ethanol and DMSO) were required to assist dissolution of farnesol in water (Koo et al. (2002) Oral Microbiol. Immunol., 17:337-343; Koo et al. (2002) Antimicrob. Agents Chemother., 46:1302-9; Koo et al. (2005) J. Dent. Res., 84:1016-20). With the micelle delivery system of the instant invention, the hydrophobic core of Pluronic® micelles (e.g., the PPO segment) acts as the hosting reservoir and readily dissolves and disperses hydrophobic drugs, such as farnesol, in water. Therefore, the benefits of this formulation also includes the prevention of irritation brought by organic solvents and, subsequently, improved patient compliance.

The size of a particular delivery system for prevention/treatment of dental biofilm is also an important factor. Due to the mechanical abrasive cleansing movement of the lips, buccal mucosa, and tongue over the surface of the teeth, the typical pattern of dental biofilm (plaque) deposition appears to be localized to the interproximal buccal and lingual surface adjacent to the gingival margin (Lamont et al. (2006). Oral Microbiology and Immunology. Washington, DC: ASM Press). The small size of the drug carrier facilitates their free access to these noted areas. Once lodged on the tooth surface, they are less likely to be removed by the above abrasive movement. As shown hereinbelow, the farnesol loaded tooth-binding micelles of the instant invention have an effective hydrodynamic diameter (D_{eff}) smaller than 100 nm, although the diameter may rise slightly when the loading in the micelle is increased (Torchilin, V. P. (2004) Cell Mol. Life Sci., 61:2549-59). Clearly, the <100 nm size of the delivery system meets the needs of the particular application, thereby leading to superior stability and efficacy in vivo.

A binding kinetic assay was performed (see Examples) to evaluate how fast and to what extent the micelles of the instant invention can bind to teeth (modeled by HA particles). In consideration of the relatively small surface area of teeth comparing to HA particles, a large excess of micelle was apply in this experiment. Fast binding kinetics is preferred for ease of use. As demonstrated hereinbelow, a positive correlation between the binding moiety content and the degree of micelle binding to HA was observed. The steepness of the slope at the beginning of the curve indicated a fast binding kinetics of micelle to the most accessible binding sites on the HA surface. Thereafter, other sites that have less accessibility would require a higher binding moiety density at the micelle surface (Hengst et al. (2007) Int. J. Pharm., 331:224-7) or longer time to achieve binding. Overall, this data indicates that even at relatively low binding moiety content, the micelle has a swift binding kinetics to the tooth surface.

The purpose of the in vitro release study from HA particle surface loaded with farnesol-encapsulating micelles was to test if the newly designed formulation would be able to provide sustained releasing kinetics of the drug and maintain a long-lasting effective drug concentration on tooth surface. As can be seen hereinbelow, the release of farnesol from the HA particle surface loaded with farnesol encapsulating micelles is rather slow. Higher drug loading seems to lead to a slower release in terms of relative percentage, but a faster release in terms of absolute amount of drug. The sustained drug releasing profile is probably due to the strong hydrophobic cohesive force between farnesol and PPO core segment of the Pluronic® (Gaucher et al. (2005) J. Control Rel., 109:169-88). Notably, the release in oral cavity is likely to be faster due to the presence of physiological factors such as saliva flow, oral protein disruption, and abrasive movements within the mouth.

In addition to dental caries, the instant invention can also be used to treat oral diseases such as periodontitis and gingivitis. The antimicrobial retained at the tooth surface would provide the adjacent infected soft tissue with sustained drug concentration for effective relief of the inflammation. Further, the delivery system could also deliver other chemicals to the surface of the tooth. These include but not limited to fragrance and dye for cosmetic purpose.

The micelles and drug delivery system of the instant invention can also specifically deliver at least one bone therapeutic agents to biominerals (e.g., bone) in a subject, applied locally or systemically. These delivery systems offer osteotropicity to bone therapeutic agents and, therefore, dramatically improve their therapeutic index. Compared to other bone-targeting technologies, the instant invention does not require chemical attachment of the chemical (e.g., drug) to the carrier and has a much higher loading capacity with the ability to load a large variety of chemicals including therapeutic or diagnostic agents.

Current technologies for the treatment of cancer bone metastasis involve non-specific administration of drugs in their free form. To be able to reach bone lesions through systemic administration, the drug is usually administered at its highest allowed concentration for a prolonged period of time. This strategy significantly increases treatment side effects. The novel drug delivery systems of the instant invention specifically bind biominerals such as bone metastasis lesion upon administration. Since the drugs are directly delivered to the bone lesion, the systemic toxic side effects of devastating cancer chemotherapeutic agents are significantly reduced and lower drug concentrations can be used. In addition, the instant invention allows for the manipulation of the degree of binding of the delivery system to a bone tissue. This adds an additional advantage of controlling the kinetics and distribution of the drug in the body.

In accordance with the present invention, compositions and methods are provided for the transport of biologically active compounds to biominerals such as bone and teeth. Specifically, the biologically active compound is contained within the hydrophobic core of micelles comprising amphiphilic copolymers. The concept of hard tissue (or biomineral)-targeting micelle applies to all amphiphilic block copolymers that can form micelles. Preferably, the amphiphilic copolymer is a biocompatible copolymer such as Pluronic®. Additionally, the amphiphilic copolymer is preferably linked to a bone and/or tooth targeting moiety. The components of the drug delivery system are described hereinbelow.

### I. Amphiphilic Copolymer

The polymer of the micelles of the instant invention may be any micelle forming polymer (e.g., block copolymer, ionic polymers). In a particular embodiment, the polymer is an amphiphilic polymer, particularly an amphiphilic block copolymer. Preferably, the amphiphilic copolymer is a biocompatible polymer, such as a Pluronic® block copolymer (BASF Corporation, Mount Olive, N.J.). Other biocompatible amphiphilic copolymers include those described in Gaucher et al. (J. Control Rel. (2005) 109:169-188. Examples of other polymers include, without limitation, Polyethylene glycol-Polylactic acid (PEG-PLA), PEG-PLA-PEG, Polyethylene glycol-Poly(lactide-co-glycolide) (PEG-PLG), Polyethylene glycol-Poly(lactic-co-glycolic acid) (PEG-PLGA), Polyethylene glycol-Polycaprolactone (PEG-PCL), Polyethylene glycol-Polyaspartate (PEG-PAsp), Polyethylene glycol-Poly(glutamic acid) (PEG-PGlu), Polyethylene glycol-Poly(acrylic acid) (PEG-PAA), Polyethylene glycol-Poly(methacrylic acid) (PEG-PMA), Polyethylene glycol-poly(ethyleneimine) (PEG-PEI), Polyethylene glycol-Poly(L-lysine) (PEG-PLys), Polyethylene glycol-Poly(2-(N,N-dimethylamino)ethyl methacrylate) (PEG-PDMAEMA) and Polyethylene glycol-Chitosan derivatives. In yet another embodiment, the polymer has the formula:

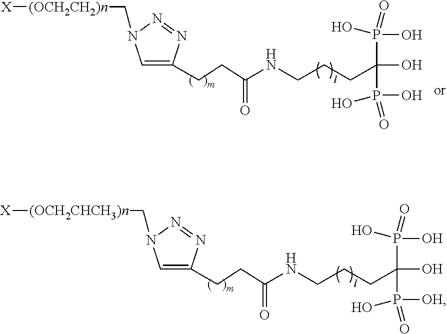

wherein n, m, and l can be any number, particularly from about 1 to about 1000, about 1 to about 100, about 1 to about 50, about 1 to about 20, or about 1 to about 5; and X is any one of as follows: hydrogen, alkyl radical, alkoxyl radical, aryl radical, ester radical, polyesters, polyacrylics, polyacrylamides, polyamides, polycarbohydrates, or any other copolymers/polymers, optionally, capped by bone targeting functional group.

The Pluronic® micelle system was selected, in part, for its simplicity and biocompatibility. Pluronic® block copolymers (listed in the U.S. and British Pharmacopoeia under the name “poloxamers”) consist of ethylene oxide (EO) and propylene oxide (PO) segments arranged in a basic A-B-A structure: EOₐ-PO_{b}-EOₐ (wherein “a” need not be the same on both sides of the PO block). This arrangement results in an amphiphilic copolymer, in which altering the number of EO units (a) and the number of PO units (b) can vary its size, hydrophilicity, and lipophilicity. A characteristic of Pluronic® copolymers is the ability to self-assemble into micelles in aqueous solutions. The noncovalent incorporation of drugs and polypeptides into the hydrophobic PO core of the Pluronic® micelle has been well-characterized and imparts to the drug increased solubility, increased metabolic stability, and increased circulation time (Kabanov and Alakhov (2002) Crit. Rev. Ther. Drug Carrier Syst., 19:1-72; Allen et al. (1999) Coll. Surfaces, B: Biointerfaces, 16:3-27; Kabanov et al. (2002) Adv. Drug Deliv. Rev., 54:223-233; U.S. Patent Application Publication No. 2006/0051317).

Pluronic® micelles conjugated with antibody to alpha 2GP have been shown to deliver neuroleptic drugs and fluorescent dyes to the brain in mice (Kabanov et al. (1989) FEBS Lett., 258:343-345; Kabanov et al. (1992) J. Contr. Release, 22:141-157). Notably, Pluronic® copolymers have also been used in combination with anticancer drugs in the treatment of multidrug resistant (MDR) cancers (Alakhov et al. (1996) Bioconjug. Chem., 7:209-216; Alakhov et al. (1999) Colloids Surf., B: Biointerfaces, 16:113-134; Venne et al. (1996) Cancer Res., 56:3626-3629). Indeed, studies have been performed on doxorubicin formulated with Pluronic® (“SP1049C”) for the treatment of adenocarinoma of esophagus and soft tissue sarcoma, both cancers with high incidence of MDR (Ranson et al. (2002) 5th international symposium on polymer therapeutics: from laboratory to clinical practice, pp. 15, The Welsh School of Pharmacy, Cardiff University, Cardiff, UK).

As stated hereinabove, the amphiphilic block copolymers (e.g., Pluronic®) can be described in terms of having hydrophilic “A” and hydrophobic “B” block segments. Amphiphilic block copolymers of the instant invention may be triblocks (A₁-B-A₂, wherein A₁ and A₂ are the same or different), diblocks (A-B or B-A), graft block copolymers (A(B)ₙ), starblocks (AₙBₘ), dendrimer based copolymers, hyper-branched (e.g., at least two points of branching) block copolymers, and Tetronic®. Preferably, the amphiphilic block copolymer is a triblock (A-B-A). The segments of the block copolymer may have from about 2 to about 1000, about 2 to about 300, or about 5 to about 100 repeating units or monomers. The ordinarily skilled artisan will recognize that in the triblock formula EOₓ-PO_{y}-EO_{z} the values of x, y, and z will usually represent a statistical average and that the values of x and z are often, though not necessarily, the same.

These block copolymers can be prepared by the methods set out, for example, in U.S. Pat. No. 2,674,619 and are commercially available from BASF under the trademark Pluronic®. Pluronic® block copolymers are designated by a letter prefix followed by a two or a three digit number. The letter prefixes (L, P, or F) refer to the physical form of each polymer, (liquid, paste, or flakeable solid). The numeric code defines the structural parameters of the block copolymer. The last digit of this code approximates the weight content of EO block in tens of weight percent (for example, 80% weight if the digit is 8, or 10% weight if the digit is 1). The remaining first one or two digits designate the molecular mass of the central PO block. To decipher the code, one should multiply the corresponding number by 300 to obtain the approximate molecular mass in daltons (Da). Therefore, Pluronic® nomenclature provides a convenient approach to estimate the characteristics of the block copolymer in the absence of reference literature. For example, the code ‘F127’ defines the block copolymer, which is in solid flake form, has a PO block of 3600 Da (12×300) and 70% weight of EO. The precise molecular characteristics of each Pluronic® block copolymer can be obtained from the manufacturer.

Over 30 Pluronic® copolymers with different lengths of hydrophilic ethylene oxide (N_{EO}) and hydrophobic propylene oxide (N_{PO}) blocks are available from BASF Corp. (see, for example, Table 1). These molecules are characterized by different hydrophilic-lipophilic balance (HLB) and CMC (Kozlov et al. (2000) Macromolecules, 33:3305-3313; see, for example, Tables 3 and 4). The HLB value, which typically falls in the range of 1 to 31 for Pluronic® block copolymers, reflects the balance of the size and strength of the hydrophilic groups and lipophilic groups of the polymer (see, for example, Attwood and Florence (1983) “Surfactant Systems: Their Chemistry, Pharmacy and Biology,” Chapman and Hall, New York) and can be determined experimentally by, for example, the phenol titration method of Marszall (see, for example, “Parfumerie, Kosmetik”, Vol. 60, 1979, pp. 444-448; Rompp, Chemistry Lexicon, 8th Edition 1983, p. 1750; U.S. Pat. No. 4,795,643). HLB values for Pluronic® polymers are available from BASF Corp. Examples of Pluronics® are provided in Tables 1.

TABLE 1

| Pluronic ® | MW ^{(a)} | N_{PO} ^{(b)} | N_{EO} ^{(b)} | HLB ^{(a)} | CMC, μM ^{(c)} |
|---|---|---|---|---|---|
| L31 | 1100 | 17.1 | 2.5 | 5 | 1180 |
| L35 | 1900 | 16.4 | 21.6 | 19 | 5260 |
| F38 | 4700 | | | 31 | |
| L42 | 1630 | | | 8 | |
| L43 | 1850 | 22.3 | 12.6 | 12 | 2160 |
| L44 | 2200 | 22.8 | 20.0 | 16 | 3590 |
| L61 | 2000 | 31 | 4.5 | 3 | 110 |
| L62 | 2500 | 34.5 | 11.4 | 7 | 400 |
| L63 | 2650 | | | 11 | |
| L64 | 2900 | 30 | 26.4 | 15 | 480 |
| P65 | 3400 | | | 17 | |
| F68 | 8400 | 29 | 152.7 | 29 | 480 |
| L72 | 2750 | | | 7 | |
| P75 | 4150 | | | 17 | |
| F77 | 6600 | | | 25 | |
| L81 | 2750 | 42.7 | 6.2 | 2 | 23 |
| P84 | 4200 | 43.4 | 38.2 | 14 | 71 |
| P85 | 4600 | 39.7 | 52.3 | 16 | 65 |
| F87 | 7700 | 39.8 | 122.5 | 24 | 91 |
| F88 | 11400 | 39.3 | 207.8 | 28 | 250 |
| L92 | 3650 | 50.3 | 16.6 | 6 | 88 |
| F98 | 13000 | 44.8 | 236.4 | 28 | 77 |
| L101 | 3800 | 58.9 | 8.6 | 1 | 2.1 |
| P103 | 4950 | 59.7 | 33.8 | 9 | 6.1 |
| P104 | 5900 | 61.0 | 53.6 | 13 | 3.4 |
| P105 | 6500 | 56.0 | 73.9 | 15 | 6.2 |
| F108 | 14600 | 50.3 | 265.4 | 27 | 22 |
| L121 | 4400 | 68.3 | 10.0 | 1 | 1 |
| L122 | 5000 | | | 4 | |
| P123 | 5750 | 69.4 | 39.2 | 8 | 44 |
| F127 | 12600 | 65.2 | 200.4 | 22 | 2.8 |

^{(a)} The average molecular weights and HLB provided by the manufacturer (BASF Co.);
^{(b)} The average numbers of EO and PO units were calculated using the average molecular weights of the blocks;
^{(c)} Critical micelle concentration (CMC) values at 37° C. were determined using pyrene probe (Kozlov et al. (2000) Macromolecules, 33: 3305-3313).

In a particular embodiment of the instant invention, the micelle comprises Pluronic® P85, P123, and/or F127, particularly P85 and/or P123. In one embodiment, the micelle comprises Pluronic® P85 and Pluronic® P123 linked to a bone or tooth targeting moiety. In a particular embodiment, 100% of the Pluronic® in the micelle is conjugated to a bone or tooth targeting moiety. In yet another embodiment, at least 5%, at least 10%, at least 20%, at least 25%, at least 30%, at least 40%, or at least 50% of the Pluronic® in the micelle is conjugated to a bone or tooth targeting moiety.

### II. Encapsulated Compounds

As stated hereinabove, the micelles of the instant invention encapsulate at least one compound (e.g., a biologically active agent). These agents or compounds include, without limitation, polypeptides, peptides, nucleic acids, synthetic and natural drugs, chemical compounds (e.g., dyes and fragrances), and lipids. The compound may be hydrophilic, hydrophobic, or amphiphilic. In a particular embodiment, the biologically active agent is hydrophobic.

When the micelles are used to deliver the compounds to teeth (e.g., to treat and/or prevent oral disease or disorders), the compound may be an antimicrobial agent. In a particular embodiment, the antimicrobial is effective against acid-tolerant and/or acid producing oral bacteria such as Lactobacilli and Streptococcus, particularly S. mutans. Antimicrobials include, without limitation, farnesol, chlorhexidine (chlorhexidine gluconate), apigenin, triclosan, and ceragenin CSA-13. In a particular embodiment, the antimicrobial is farnesol. In another embodiment, the antimicrobial is an antibiotic such as, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), metronidazole, polypeptides (e.g., colistin), and derivatives thereof.

Farnesol (3,7,11-trimethyl-2,6,10-dodecatrien-1-ol), a recently identified anti-caries natural product found in propolis (Koo et al. (2002) Oral Microbiol. Immunol., 17:337-343; Koo et al. (2002) Antimicrob. Agents Chemother., 46:1302-9; Koo et al. (2005) J. Dent. Res., 84:1016-20), was used as a model drug in the studies presented hereinbelow. It was found that the farnesol formulation is capable of providing complete inhibition of biofilm formation mediated by S. Mutans UA159.

Oral diseases and disorders that can be treated and/or prevented by the administration of the micelles of the instant invention include, without limitation, caries, gingivitis, periodontitis, periodontitis-associated bone loss, dentin hypersensitivity, oral mucosal disease, oral mucositis, vesiculo-erosive oral mucosal disease, stained/discolored teeth, dry mouth, and halitosis. In a particular embodiment, the encapsulated compound is an antimicrobial, anti-inflammatory, menthol, a fragrant agent (e.g., limonene, orange oil), a flavoring agent, cooling agent, fluoride, vitamins, neutraceuticals, tooth whitening agents, tooth coloring agents, bleaching or oxidizing agents, thickening agents, and sweetening agents. Examples of such agents can be found, for example, in U.S. Patent Application Publication No. 2006/0286044 and PCT/EP2005/009724.

When the micelles are used to deliver the biologically active agent to bone (e.g., to treat and/or prevent a bone related disease or disorder), the biologically active agent may be a bone related therapeutic agent. A “bone related therapeutic agent” refers to an agent suitable for administration to a patient that induces a desired biological or pharmacological effect such as, without limitation, 1) increasing bone growth, 2) preventing an undesired biological effect such as an infection, 3) alleviating a condition (e.g., pain or inflammation) caused by a disease associated with bone, and/or 4) alleviating, reducing, or eliminating a disease (e.g., cancer) from bone. The bone related therapeutic agent possesses a bone anabolic effect and/or bone stabilizing effect. Bone related therapeutic agents include, without limitation, cathepsin K inhibitor, metalloproteinase inhibitor, prostaglandin E receptor agonist, prostaglandin E1 or E2 and analogs thereof, parathyroid hormone and fragments thereof, glucocorticoids (e.g., dexamethasone) and derivatives thereof, chemotherapeutic agents, and statins (e.g., simvastatin).

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, and Pseudomonas exotoxin); taxanes; alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes (e.g., cisplatin, carboplatin, tetraplatin, ormaplatin, thioplatin, satraplatin, nedaplatin, oxaliplatin, heptaplatin, iproplatin, transplatin, and lobaplatin); bioreductive alkylators such as mitomycin, procarbazine, dacarbazine and altretamine); DNA strand-breakage agents (e.g., bleomycin); topoisomerase II inhibitors (e.g., amsacrine, menogaril, amonafide, dactinomycin, daunorubicin, N,N-dibenzyl daunomycin, ellipticine, daunomycin, pyrazoloacridine, idarubicin, mitoxantrone, m-AMSA, bisantrene, doxorubicin (adriamycin), deoxydoxorubicin, etoposide (VP-16), etoposide phosphate, oxanthrazole, rubidazone, epirubicin, bleomycin, and teniposide); DNA minor groove binding agents (e.g., plicamydin); antimetabolites (e.g., folate antagonists such as methotrexate and trimetrexate); pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); and tubulin interactive agents (e.g., vincristine, vinblastine, and paclitaxel (Taxol)).

Bone disease and disorders that can be treated and/or prevented by the instant invention include, without limitation, bone cancer, osteoporosis, osteomyalitis, osteopenia, bone fractures, bone breaks, Paget's disease (osteitis deformans), bone degradation, bone weakening, skeletal distortion, low bone mineral density, scoliosis, osteomalacia, osteomyelitis, osteogenesis imperfecta, osteopetrosis, enchondromatosis, osteochondromatosis, achondroplasia, alveolar bone defects, spine vertebra compression, bone loss after spinal cord injury, avascular necrosis, fibrous dysplasia, periodontal disease, hyperparathyroidism (osteitis fibrosa cystica), hypophosphatasia, fibrodysplasia ossificans progressive, and pain and inflammation of the bone.

In a particular embodiment, the micelles of the instant invention may be used with a bone graft. In a particular embodiment, the micelles may comprise at least one bone related therapeutic agent (e.g., growth factor) and/or at least one antimicrobial. In a particular embodiment, the bone related therapeutic agent is prostaglandin E1 or E2 or a statins (e.g., simvastatin). The micelles may be administered with the bone graft (e.g., applied to the graft or administered at the same time) and/or after the bone graft.

### III. Biomineral Targeting Moieties

The micelles of the instant invention include at least one targeting moiety which is used to direct the delivery system to a specific tissue, such as bone, cartilage, or tooth. Illustrative examples of targeting moieties include, but are not limited to, folic acid, mannose, bisphosphonates (e.g., alendronate), quaternary ammonium groups, peptides (e.g., peptides comprising about 2 to about 100 (particularly 6) D-glutamic acid residues, L-glutamic acid residues, D-aspartic acid residues, L-aspartic acid residues, D-phosphoserine residues, L-phosphoserine residues, D-phosphothreonine residues, L-phosphothreonine residues, D-phosphotyrosine residues, and/or L-phosphotyrosine residues), tetracycline and analogs or derivatives thereof, sialic acid, malonic acid, N,N-dicarboxymethylamine, 4-aminosalicyclic acid, and/or antibodies or fragments or derivatives thereof specific for bone or tooth (e.g., Fab, humanized antibodies, and/or single chain variable fragment (scFv)). In a particular embodiment, the targeting moiety is alendronate.

Alendronate, a bisphosphonate, has a high affinity for hydroxyapatite crystals (the main component of tooth enamel), and has been used clinically for the treatment of osteoporosis for many years (Russell, R. G. (2007) Pediatrics 119(Suppl 2):S150-62).

The targeting moiety may be linked to the copolymer (e.g., the copolymer backbone) via covalent or physical bonds (linkages). The linkage between the targeting moiety and the amphiphilic polymer can be a direct linkage between a functional group at a termini of the polymer and a functional group on the targeting moiety. Optionally, the spacers/linker between a targeting moiety and the polymer backbone may be cleaved upon a stimulus including, but not limited to, changes in pH, presence of a specific enzyme activity (i.e., the linker comprises an amino acid sequence cleavable by a protease), presence of reductases (i.e., linker comprises disulfide bond), changes in oxygen levels, etc. In other words, the linker may be nondegradable or degradable (e.g., substantially cleaved). A biodegradable linker (e.g. L-Asp hexapeptide) may be used to prevent any possible accumulation of the micelles post drug release.

As an example, alendronate was conjugated to the chain termini of P123 by Cu-catalyzed Huisgen 1,3-dipolar cycloaddition of azides and terminal alkynes (HDC reaction). The HDC reaction has the merits of high efficiency, reliability and tolerance of reaction conditions. It can be performed over a wide range of temperatures (0-160° C.), in a variety of solvents (including water), and over a wide range of pH values (e.g., 5 through 12) (Hein et al. (2008) Pharm. Res., 25:2216-30). The 1,2,3-triazoles linker it yields is extremely water soluble and is stable against hydrolysis under typical biological conditions (Kolb et al. (2003) Drug Discov. Today 8:1128-37), which prevents the premature loss of the drug from teeth surface due to the failure of the connection between micelle and the binding moiety.

### IV. Therapy

The chemical containing micelles described herein will generally be administered to a patient as a pharmaceutical preparation. The term “patient” as used herein refers to human or animal subjects. These micelles may be employed therapeutically, under the guidance of a physician. In addition, the micelles may also be used to deliver cosmetic compounds or nutraceuticals.

The compositions of the instant invention comprise 1) at least one of the micelles described hereinabove comprising at least one biologically active agent and 2) optionally, at least one pharmaceutically acceptable carrier.

The dose and dosage regimen of the compositions according to the invention that is suitable for administration to a particular patient may be determined by a physician considering the patient's age, sex, weight, general medical condition, and the specific condition for which the composition is being administered and the severity thereof. The physician may also take into account the route of administration of the composition, the pharmaceutical carrier with which the micelles is to combined, and the micelle's biological activity.

Compositions of the instant invention may be administered by any method such as intravenous injection into the blood stream, oral administration, or by subcutaneous, intramuscular or intraperitoneal injection. Pharmaceutical preparations for injection are known in the art. If injection is selected as a method for administering the composition, steps must be taken to ensure that sufficient amounts of the molecules reach their target cells to exert a biological effect.

Pharmaceutical compositions containing a conjugate of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral, direct injection, intracranial, and intravitreal. In preparing the amphiphilic polymer-protein conjugate in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which solid pharmaceutical carriers are employed. If desired, tablets may be sugar-coated or enteric-coated by standard techniques. Injectable suspensions may also be prepared, in which case appropriate liquid carriers, suspending agents and the like may be employed. Additionally, the conjugate of the instant invention may be administered in a slow-release matrix. For example, the conjugate may be administered in a gel comprising unconjugated poloxamers.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of the composition of the instant invention may be determined by evaluating the toxicity of the molecules in animal models. Various concentrations of the pharmaceutical preparations may be administered to mice, and the minimal and maximal dosages may be determined based on the beneficial results and side effects observed as a result of the treatment. Appropriate dosage unit may also be determined by assessing the efficacy of the pharmaceutical preparation treatment in combination with other standard drugs. The dosage units of the pharmaceutical preparation may be determined individually or in combination with each treatment according to the effect detected.

The pharmaceutical preparation may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The composition of the instant invention may be used to treat and/or prevent caries. Treating caries may include administration of the compositions of the present invention to a subject suffering from caries for the purpose of reducing the amount of cariogenic bacteria such as Streptococcus mutans and/or for completely depleting Streptococcus mutans from the oral cavity, mouth, and/or teeth. The prevention of caries includes prophylaxis of caries. The compositions of the instant invention may be administered to subjects who have are at risk for encountering cariogenic bacteria such as Streptococcus mutans (e.g., have not encountered cariogenic bacteria and/or do not currently have cariogenic bacteria in the oral cavity). The compositions may be administered to infants or children for prophylaxis of caries since their oral cavity is normally free of Streptococcus mutans.

When used to treat and/or prevent oral disease or disorders, the micelles of the instant invention may be contained within a composition comprising at least one orally acceptable carrier (i.e., a pharmaceutically acceptable carrier which can be used to apply the composition to the oral cavity in a safe and effective manner). Preferably, the composition of the present invention is for use in oral applications. Accordingly, the composition may be in the form of a mouthwash, toothpaste, dentifrice (paste, liquid, or powder), dental floss coating, dental film, tooth powder, topical oral gel, mouth rinse, denture product, mouthspray, lozenge, oral tablet, chewable tablet, or chewing gum. Such compositions may further comprise other oral active agents such as, without limitation, chelating agents, fluoride, teeth whitening agents, tooth coloring agents (including non-natural colors), bleaching or oxidizing agents, cooling agent, vitamins, neutaceuticals, thickening agents, humectants, flavouring agents, fragrant agents, sweetening agents, and other antimicrobial agents. These agents may be encapsulated within the micelles or contained within the composition comprising the micelles.

### Definitions

The following definitions are provided to facilitate an understanding of the present invention:

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water).

As used herein, the term “hydrophilic” means the ability to dissolve in water.

As used herein, the term “amphiphilic” means the ability to dissolve in both water and lipids. Typically, an amphiphilic compound comprises a hydrophilic portion and a hydrophobic portion.

The term “substantially cleaved” refers to the cleavage of the amphiphilic polymer from the protein of the conjugates of the instant invention, preferably at the linker moiety. “Substantial cleavage” occurs when at least 50% of the conjugates are cleaved, preferably at least 75% of the conjugates are cleaved, more preferably at least 90% of the conjugates are cleaved, and most preferably at least 95% of the conjugates are cleaved.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxilliary agent, filler, disintegrant, lubricating agent, binder, stabilizer, preservative or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

A “therapeutically effective amount” of a compound or a pharmaceutical composition refers to an amount effective to prevent, inhibit, or treat the symptoms of a particular disorder or disease.

The following examples provide illustrative methods of practicing the instant invention, and are not intended to limit the scope of the invention in any way. While certain of the following examples specifically identify a certain type of Pluronic® block copolymer (e.g., Pluronic® P85 and P123), the use of any amphiphilic block copolymer or Pluronic® is within the scope of the instant invention.

### EXAMPLE 1

### Materials and Methods

### Chemicals

Alendronate (ALN) was purchased from Ultratech India Ltd. (New Mumbai, India). Farnesol was obtained from TCI America (Portland, Oreg.). Hydroxyapatite particle (HA, DNA grade Bio-Gel HTP gel) was purchased from Bio-Rad (Hercules, Calif.). Hydroxyapatite discs were purchased from Clarkson Chromatography Products, Inc. (South Williamsport, Pa.). LH-20 resin was purchased from GE Healthcare (Piscataway, N.J.). Pluronic® copolymers (P85 and P123) were obtained from BASF (Florham Park, N.J.). All other reagents and solvents, if not specified, were purchased from either Fisher Scientifics (Pittsburgh, Pa.) or Acros Organics (Morris Plains, N.J.).

### Methods

¹H NMR spectra were recorded on a Varian Inova Unity 500 NMR Spectrometer. Measurements were conducted at room temperature in 5 mm NMR tubes. UV-Visible spectra were measured on a Shimadzu UV-1601PC UV-Visible Spectrophotometer. Electrophoretic mobility measurements were performed using a ZetaPlus analyzer (Brookhaven Instrument Co.) with a 30 mW solid-state laser operating at a wavelength of 635 nm. ζ-potential (ζ) of the micelles was calculated from the electrophoretic mobility values using Smoluchowski equation. Effective hydrodynamic diameters (D_{eff}) of the micelles were measured by photon correlation spectroscopy (DLS) in a thermostatic cell at a scattering angle of 90° using the same instrument equipped with a Multi Angle Sizing Option (BI-MAS). All measurements were performed at 25° C. Software provided by the manufacturer was used to calculate the size of the particles and polydispersity indices. The diameters mean values were calculated from the measurements performed in triplicate. An Agilent 1100 HPLC system with a quaternary pump and degasser, an autosampler, a fluorescence detector and a diode-array based UV detector was used for drug release analysis.

### Synthesis of pentynoic acid 2,5-dioxo-pyrrolidin-1-yl ester (1)

4-Pentynoic acid (2.0 g, 20 mmol) was first dissolved in CH₂Cl₂ (80 mL). N-Hydroxysuccinimide (NHS, 2.54 g, 22 mmol) and 1-ethyl-3-(3-dimethylaminopropyl) carbodiimide hydrochloride (EDC, 4.22 g, 22 mmol) were then added to the solution. After overnight reaction at room temperature with stirring, the reaction mixture was concentrated and the pure product was obtained with silica gel column (hexane:ethyl acetate=2:1). Yield: 85%. ¹H NMR (CDCl₃) δ (ppm) 2.88-2.83 (m, 6 H), 2.60 (td, J₁=2.44 Hz, J₂=7.81 Hz, 2H), 2.04 (t, J=2.44 Hz, 1H).

### Synthesis of 1-hydroxy-4-pent-4-ynamidobutane-1,1-diyldiphosphonic acid (2)

Alendronate (3.15 g, 10 mmol) was dissolved in water or PBS (60 mL, pH adjusted to 7.0). Three batches of pentynoic acid 2,5-dioxo-pyrrolidin-1-yl ester (compound 1, 0.78 g×3, a total of 12 mmol, all in acetonitrile) were then added dropwise into this solution during the period of 12 hours at 4 hour intervals. The reaction solution was concentrated and precipitated into ethanol for 3 times to give the pure product. Yield: 90%. ¹H NMR (D₂O) δ (ppm) 3.20 (t, J=6.84 Hz, 2H), 2.44 (m, 4H), 2.37 (t, J=2.44 Hz, 1H), 1.90 (m, 2H), 1.80 (m, 2H).

### Synthesis of p-toluenesulfonyl terminated Pluronic® 123 (Tos-P123, 3)

P123 (10.5 g, 2 mmol) was dried by azeotropic evaporation with toluene (3×50 mL) and dissolved under argon in anhydrous dichloromethane (DCM, 20 mL) together with 4-dimethylaminopyridine (DMAP, 0.122 g, 1 mmol) and triethylamine (TEA, 2.02 g, 20 mmol). The reaction mixture was cooled to 0° C. and p-toluenesulfonyl chloride (3.18 g, 20 mmol) was added. After overnight reaction at room temperature, the mixture was washed by hydrochloric acid (0.1 M, 2×10 mL), water (2×10 mL), brine (2×10 mL) and then dried over anhydrous magnesium sulfide. After removal of the solvent under reduced pressure and drying over anhydrous magnesium sulfide, the crude product was further purified by LH-20 column. Yield: 60%. ¹H NMR (DMSO-d₆) δ (ppm) 7.79 (d, J=8.29 Hz), 7.48 (d, J=8.29 Hz), 4.11 (t, J=4.88 Hz), 3.65-3.43 (m), 1.04 (d, J=4.39 Hz).

### Synthesis of azide terminated Pluronic® 123 (Azido-P123, 4)

Tos-P123 (1.64 g, 0.27 mmol) was dissolved in dimethylformamide (DMF, 20 mL). Sodium azide (0.176 g, 2.7 mmol) was then added. The reaction proceeded with stirring at 100° C. for 2 days. After filtration and solvent removal, the crude product was dissolved in DCM, washed with water (2×10 mL) and brine (2×10 mL) and then dried over anhydrous magnesium sulfide. After removal of the solvent under vacuum, the product was obtained. Yield: 96.2%. ¹H NMR (DMSO-d₆) δ (ppm) 3.61 (t, J=4.88 Hz), 3.56-3.43 (m), 1.04 (d, J=4.39 Hz).

### Synthesis of Pluronic® 123-alendronate conjugate (ALN-P123, 5)

Azido-P123 (2.9 g, 0.5 mmol), 1-hydroxy-4-pent-4-ynamidobutane-1,1-diyldiphosphonic acid (0.395 g, 1 mmol) were dissolved in EtOH/H₂O solution (1/1, 15 mL). Sodium ascorbate (0.198 g, 1 mmol) and copper sulfide pentahydrate (25 mg, 0.1 mmol) were then added. The reaction mixture was allowed to stir for 3 days at room temperature. After removal of the solvent, the product was acidified and purified with LH-20 column using methanol as the eluent. Yield: 70%. ¹H NMR (D₂O) δ (ppm) 7.81 (s), 3.93 (t, J=4.90 Hz), 3.80-3.39 (m), 3.14 (t, J=6.83 Hz), 1.86 (m), 1.75 (m), 1.12 (d, J=7.81 Hz).

### Preparation and Characterization of Tooth-Binding Micelle

Varying amounts of farnesol (20, 40, 70, or 100 mg) were added to 10 mL of 2% (w/w) Pluronic®-water solution (different ALN-P123 to P85 ratio). The mixture was subjected to vortex mixing for 30 seconds and placed at 37° C. overnight with gentle shaking to equilibrium. The resulting micelle solutions were filtered (0.45 μm filter) before measurement of ζ-potential and effective hydrodynamic diameters (D_{eff}).

### Binding Kinetics of Tooth-Binding Micelle on HA Particles

The micelle solution (0.2 mL/tube, containing 4 mg/mL of farnesol) was mixed with HA particles (20 mg/tube) in centrifuge tubes. The tubes were placed on a Labquake® rotator to allow binding at room temperature. At each predetermined time points, 3 tubes were taken out, centrifuged (12,000 rpm, 0.5 minutes), and 100 μL of the supernatant was collected. The collected samples were then diluted 100 times and analyzed by HPLC. Agilent C₁₈ reverse-phase column (4.6×250 mm, 5 μm) was used with a mobile phase of acetonitrile/water (80:20, v/v) at a flow rate of 1 ml/minute. The UV detection was set at 210 nm. The amount of farnesol bound to HA particles via the micellar formulation was calculated by subtracting the amount of farnesol left in the supernatant from the initial amount of drug added.

In Vitro Release of Farnesol from Tooth-Binding Micelle Immobilized on HA Particles

The micelle solution (1 mL, ALN-P123 to P85 ratio=1/4) was mixed with HA particles (100 mg) for 60 minutes to allow binding of the micelles to HA. The mixture was then centrifuged and HA particles were washed 3 times with water to remove unbound micelles. The micelle-loaded HA particles were then resuspended in 1 mL of releasing medium (0.1 M PBS, pH=7.4) and placed on a Labquake® rotator to allow drug release at 37° C. At predetermined time intervals, samples were centrifuged and the supernatant was removed and replaced with 1 mL of fresh medium, then resuspended. The collected supernatant (1 mL) was mixed with acetonitrile (0.5 mL), filtered (0.2 μm) and analyzed with HPLC. At the end point of the experiment, HA particles were washed 3 times with acetonitrile to release the remaining drug, and the total amount of farnesol loaded on HA particles was calculated.

### In Vitro Inhibition of Streptococcus Mutans (S. Mutans) Biofilm Growth on HA Discs

S. Mutans UA159 was used in this study. S. Mutans were stored frozen at −80° C. A fresh culture was prepared before each experiment. A single colony of S. Mutans was inoculated into 3 mL of THYE (Todd-Hewitt Yeast Extract) medium and allow to growth statically overnight at 37° C. with 5% CO₂. The overnight culture was diluted to a density of 5×10⁴ CFU/ml with chemically defined biofilm media (CDM) (Biswas et al. (2007) J. Bacteriol., 189:6521-6531).

Autoclaved HA discs (7 mm×1.8 mm) were incubated with different micelle solutions, a farnesol solution in ethanol, or CDM in a 24-well plate for 1 hour to achieve maximum loading. The discs were then removed from the wells and vortex washed twice with saline for 10 seconds. The discs were subsequently washed with culture media to remove unbounded micelle. For the farnesol ethanol solution group, discs were washed twice with ethanol and then washed with saline. The HA discs were then transferred to 1 mL of diluted S. Mutans, and cultured statically for 48 hours to allow biofilm growth at 37° C. with 5% CO₂.

On day three post bacteria inoculation, the HA discs were dip-washed 3 times with THYE media to remove loosely attached bacteria and then placed in 1 mL of THYE media. The surfaces of HA discs were gently scraped with a sterile spatula to harvest adherent cells. The cell suspensions were subjected to vortex mixing for 10 seconds and then sequentially diluted at a 1:10 ratio 5 times (for the blank control group, empty micelle group, non-binding micelle group, and farnesol ethanol solution groups). The last 3 dilutions (10 μL each) were plated on THYE agar and incubated for 48 hours at 37° C. with 5% CO₂. For teeth-binding micelle groups, 100 μL of undiluted solution or 10 μL of either undiluted solution or 10 times diluted solution were plated on THYE agar and incubated for 48 hours at the same conditions. Biofilm assays were performed in triplicate in three different experiments.

### Results

### Preparation and Characterization of the Tooth-Binding Micelle

The multi-step synthesis of Pluronic® 123-alendronate conjugate (ALN-P123) is important for the successful generation of tooth-binding micelle. Each reaction step was accomplished with reasonable yields of at least 60%. After micelle preparation, effective hydrodynamic diameters (D_{eff}) and ζ-potential of the micelles of different preparations were measured by photon correlation spectroscopy (DLS) (Table 2). Both empty micelles and farnesol loaded non-binding micelles have the biggest particle size which was around 100 nm. Farnesol loaded tooth-binding micelles have a relatively smaller size, which increases as the farnesol loading was raised. In the loading range tested, however, the D_{eff} of farnesol loaded tooth-binding micelles does not exceed 100 nm.

TABLE 2
D_{eff} and ζ of different micelle solutions.

| | P85 | ALN-P123 | Farnesol | D_{eff} | Z |
|---|---|---|---|---|---|
| Entry | (%) | (%) | (%) | (nm) | (mV) |
| 1 | 1.8 | 0.2 | 0 | 99.5 ± 5.8 | — |
| 2 | 1.8 | 0.2 | 0.4 | 45.1 ± 0.7 | −27.06 |
| 3 | 1.8 | 0.2 | 0.7 | 53.0 ± 0.6 | — |
| 4 | 1.8 | 0.2 | 1.0 | 68.2 ± 1.5 | — |
| 5 | 2.0 | 0 | 1.0 | 99.5 ± 5.8 | −0.37 |
| 6 | 1.95 | 0.05 | 0.4 | 95.5 ± 1.6 | −8.53 |
| 7 | 1.9 | 0.1 | 0.4 | 83.7 ± 2.6 | −10.25 |

Binding kinetics of Tooth-Binding Micelle to HA Particles

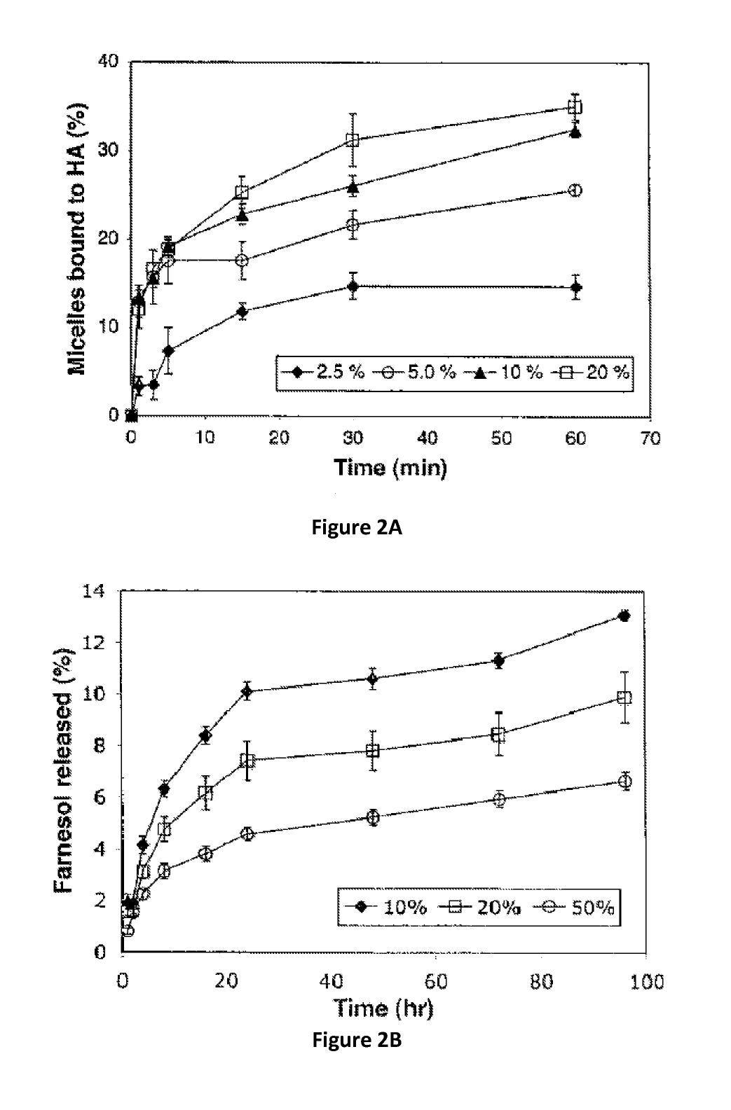

As shown in FIG. 2A, the amount of binding moiety (alendronate) presented on micelle surface significantly affected micelle binding efficiency. The amount of micelle bound to HA particles was enhanced by increasing the content of ALN-P123. All preparations containing 0.1%, 0.2% or 0.4% of ALN-P123 bind quickly to the HA particles and reach binding plateau within 5 minutes.

### In Vitro Release of Teeth Binding Micelles

The in vitro release profile of farnesol from tooth-binging micelles that bound to HA powders was evaluated over a 4-day period. As seen in FIG. 2B, a gradual releasing profile of farnesol from the tooth-binding micelles that bound HA was observed.

### Inhibition of S. Mutans Biofilm Growth on HA Discs

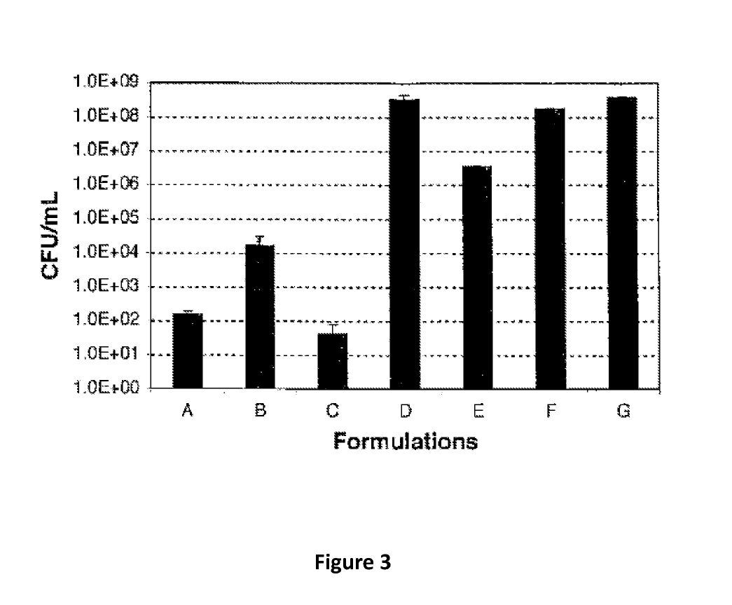

As shown in FIG. 3, all tooth-binding micelle groups showed high levels of biofilm inhibition when compared to blank control. Indeed, a 4 orders of magnitude decrease in CFU/biofilm was observed. Non-binding micelles showed a rather weak inhibitatory effect, probably due to non-specific binding of the micelle. Farnesol solution did not show inhibition as the drug was washed away in the washing practice due to lack of retention on HA.

Thus, a novel tooth-binding micelle delivery system for the delivery of an anti-caries agent (e.g., farnesol) has been successfully developed. It could bind to teeth surfaces quickly and release the drug in a sustained manner. Bacterial biofilm studies revealed that the formulation could effectively inhibit S. mutans biofilm growth on HA disc.

### EXAMPLE 2

### Preparation of Bone-Targeting Micelles

45 mg P123, 5 mg ALN-P123, and 1 mg Rhodamine B labeled P123 (RB-P123) were dissolved in 2 mL methanol in a flask. The solvent was evaporated in vacuum to yield a polymeric film on the wall of the flask. The thin polymeric film formed was then hydrated with a 10 mM phosphate buffered saline solution (PBS, pH 7.4) at 50° C. to set the micelles.

### Binding Potential and Rate of Bone-Targeting Micelles on Hydroxyapatite (HA)

The micelles were prepared as the method mentioned above for bone-targeting micelles: 0.9% P123, 0.1% ALN-P123, 0.02% RB-P123; for controls: 1% P123, 0.02% RB-P123). HA (100 mg) was then added into 1 mL of the solution. The mixtures were allowed to be gently agitated for 1, 5, 10, or 30 minutes at room temperature. HA was removed by centrifugation (10000 rpm, 0.5 minutes). The spectra of the supernatant was recorded on a UV-Visible spectrophotometer and compared with that of the initial micelle solution. Micelle containing RB-P123 but no ALN-P123 and RB were used as controls in this experiment.

### Preparation of Drug Loaded Bone-Targeting Micelles

135 mg P123, 15 mg ALN-P123, and 15 mg simvastatin were dissolved in 2 mL methanol in a flask. The solvent was evaporated in a vacuum to yield a polymeric film on the wall of the flask. The thin polymeric film formed was then hydrated in 3 mL phosphate buffered saline solution (PBS, 10 mM, pH 7.4) at 50° C. The suspension was then filtered using a syringe through a 0.22 μm filter to remove uncapsulated simvastatin. The drug content in micelles was determined by HPLC: Agilent C18 reverse-phase column (4.6×250 mm, 5 μm); mobile phase: acetonitrile/water (70:30, v/v) at a flow rate of 1 ml/min; UV detection at 335 nm.

### Drug Loading of Bone-Targeting Micelles on HA Surface

250 mg HA was added into 1 mL simvastain loaded micelles. The mixture was shaken for at least 30 minutes, followed by filtration and drying to give the simvastatin loaded HA. 100 mg simvastatin loaded HA were extracted with methanol/water solution for 5 times and analyzed by HPLC: Agilent C18 reverse-phase column (4.6×250 mm, 5 μm); mobile phase: acetonitrile/water (70:30, v/v) at a flow rate of 1 ml/minute; UV detection at 235 nm.

### Results

### Binding Potential and Binding Kinetics of Bone-Targeting Micelles to HA

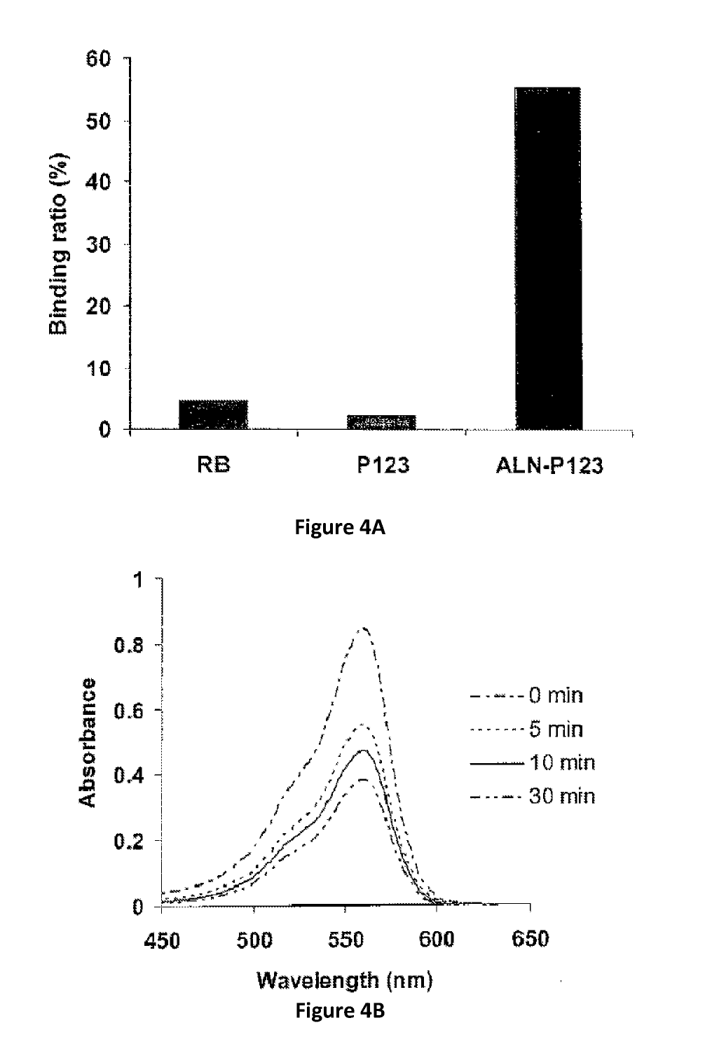

After incubation with HA, the amount of ALN-P123 not bound to HA was measured with UV/Vis spectrophotometer. Compared to the original solution, the UV absorbance at 565 nm for ALN-P123 decreased to 55% of the original after 30 minutes of incubation, which indicated large portion of the ALN-P123 bound to H4 surface via the bisphosphonate moiety (FIG. 4A). On the other hand, micelles without bone-targeting moiety and RB just slightly bound to HA, potentially due to non-specific binding to HA surface. Repeated washing of the HA with water yielded white powder except for those treated with ALN-P123, which remained pink. The binding of the conjugates to the surface HA was observed to occur very quickly. The binding of ALN-P123 almost reached a plateau in 10 minutes with 45% of the conjugate bound to HA (FIG. 4B). Prolonged incubation of the ALN-P123 with HA for 30 minutes led to an ultimate binding equilibrium of 55% bound. This outstanding HA-binding ability indicates a strong osteotropicity of the novel micelles and the ability for the tissue-specific delivery of therapeutic agents to the skeleton. This rapid binding to HA (model bone) has particular application to systemic delivery of statins, which have robust bone anabolic properties, but are quickly cleared from the circulation by the liver.

### Drug Loading of Bone-Targeting Micelles on HA Surface

The results of the drug loading of bone-targeting micelles on HA surface are shown in Table 3. The drug loaded targeting micelles can bind to HA very efficiently. Significantly, the non-targeting micelles cannot bind to HA.

TABLE 3
Drug loading results.

| Samples | [Simvastatin] |
|---|---|
| Micelles with 0.5% ALN-P123, 4.5% P123, | 3.37 mg/ml |
| and 0.5% simvastatin | |
| Micelles with 5% P123 and 0.5% | 1.80 mg/ml |
| simvastatin | |
| Micelles with 2.25% ALN-P123, 4.25% P85 | 2.42 mg/ml |
| and 8.5% P123, and 1.5% simvastatin | |
| Micelles with 5% P85 and 10% P123 and | 1.43 mg/ml |
| 1.5% simvastatin | |
| Simvastatin loaded ALN-P123 micelles | 4 mg/ml |
| (preparation 1) on HA | |
| Simvastatin loaded P123 micelles | No detection |
| (preparation 2) on HA | |

### In Vitro Release of Bone-Targeting Micelles on HA Surface

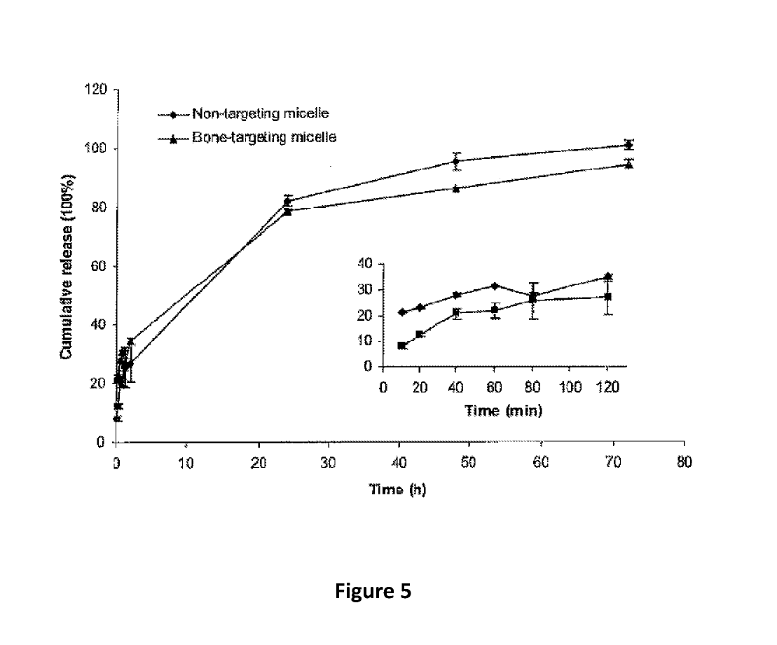

The micelles were prepared with the method described above (for bone-targeting micelles: 2.25% ALN-P123, 4.25% P85, 8.5% P123, and 1.5% simvastatin; for non-targeting micelles, 5% P85, 10% P123, and 1.5% simvastatin). Bone-targeting micelles or non-targeting micelles (50 mg, 2mL) were mixed with excessive hydroxyapatite (HA) (500 mg) for 30 minutes to allow full binding of bone-targeting micelles to HA. Then the mixture was sealed in a dialysis bag (with a MW cutoff of 12,000). The bag was incubated in 20 mL release medium (0.1 M PBS, pH 7.4, containing 2.5% P123 to maintain sink condition) with gentle shaking (50 rpm) at 37° C. At predetermined time intervals, 0.5 mL of release medium was collected and replaced with fresh medium. Collected samples were mix with 0.5 mL acetonitrile, filtered through a 0.2 μm filter and analyzed by HPLC (mobile phase: acetonitrile:water, 70/30, v/v). Results are shown in FIG. 5. Both bone-targeting micelles and non-targeting micelles had similar release profiles where most of the drug (around 80%) was released within 24 hours.

### In Vivo Bone Anabolic Effect of Bone-Targeting Micelles in Mice

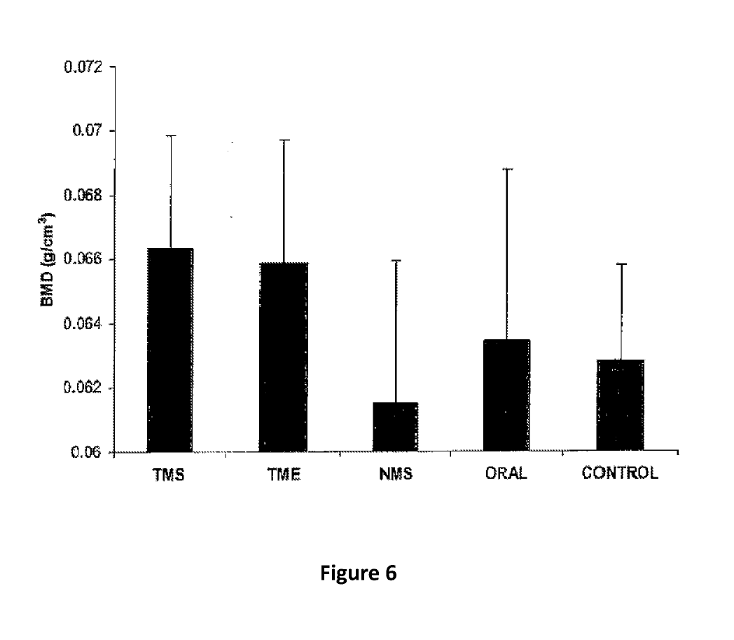

The micelles were prepared with the method described above (for bone-targeting micelles: 2.25% ALN-P123, 4.25% P85 and 8.5% P123, and 1.5% simvastatin; for non-targeting micelles: 5% P85 and 10% P123, and 1.5% simvastatin). Mice (retired breeders) were randomly separated into 5 groups and were given simvastatin loaded bone-targeting micelles (TMS), empty bone-targeting micelles (TME), simvastatin loaded non-targeting micelles (NMS), simvastatin solution (ORAL), or not treated (CONTROL). Micelles were given to mice through tail vein injection every 4 days at the dose of 40 mg simvastatin per Kg body weight for 28 days. Simvastatin solution (1 mg/mL, 0.5% methylcellulose solution) was given through oral gavage every day at the dose of 10 mg simvastatin per Kg body weight for 28 days. After 28 days, mice were sacrificed and tibias (×2) were separated for BMD measurement using P-Dexa. Results are shown in FIG. 6. Both simvastatin loaded bone-targeting micelles (TMS group) and empty bone-targeting micelles (TME group) significantly increased BMD when compared to control (P<0.05). The TMS group showed a higher BMD than the TME group. Oral gavage of simvastatin solution and tail vein injection of non-targeting micelles were not able to significantly increase BMD (P>0.05).

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for treating or inhibiting an oral disease or disorder in a subject, said method comprising administering to said subject a composition comprising:
a) micelles comprising i) at least one amphiphilic block copolymer linked to at least one tooth targeting moiety and ii) at least one encapsulated compound; and
b) at least one pharmaceutically acceptable carrier.

2. The method of claim 1, wherein said oral disease or disorder is dental caries.

3. The method of claim 1, wherein said amphiphilic block copolymer comprises at least one poly(ethylene oxide) (EO) segment and at least one poly(propylene oxide) (PO) segment.

4. The method of claim 3, wherein said amphiphilic block copolymer has the formula: EOₓ-PO_{y}-EO_{z},wherein x, y, and z have values from about 2 to about 300.

5. The method of claim 1, wherein the amphiphilic block copolymer is linked to the tooth targeting moiety by a cleavable linker.

6. The method of claim 1, where said encapsulated compound is selected from the group consisting of antimicrobial agent, anti-inflammatory agent, menthol, fragrant agent, flavoring agent, cooling agent, fluoride, vitamin, neutraceutical, tooth whitening agent, tooth coloring agent, bleaching or oxidizing agent, thickening agent, and sweetening agent.

7. The method of claim 2, wherein said encapsulated compound is an antimicrobial agent.

8. The method of claim 7, wherein said antimicrobial agent is farnesol.

9. The method of claim 1, wherein said tooth targeting moiety is alendronate.

10. The method of claim 1, wherein said composition is selected from the group consisting of a mouthwash, toothpaste, dentifrice, film, dental floss coating, tooth powder, topical oral gel, mouth rinse, denture product, mouthspray, lozenge, oral tablet, chewable tablet, and chewing gum.

11. A method for treating or inhibiting a bone disease or disorder in a subject, said method comprising administering to said subject a composition comprising:
a) micelles comprising i) at least one amphiphilic block copolymer linked to at least one bone targeting moiety and ii) at least one bone related therapeutic agent; and
b) at least one pharmaceutically acceptable carrier.

12. The method of claim 11, wherein said amphiphilic block copolymer comprising at least one poly(ethylene oxide) (EO) segment and at least one poly(propylene oxide) (PO) segment.

13. The method of claim 11, wherein said amphiphilic block copolymer has the formula: EOₓ-PO_{y}-EO_{z}, wherein x, y, and z have values from about 2 to about 300.

14. The method of claim 11, wherein the amphiphilic block copolymer is linked to the bone targeting moiety by a cleavable linker.

15. The method of claim 11, wherein said bone related therapeutic agent is a chemotherapeutic agent.

16. The method of claim 11, wherein said bone targeting moiety is alendronate.

17. A composition comprising:
a) micelles comprising i) at least one amphiphilic block copolymer linked to at least one hard tissue targeting moiety and ii) at least one biologically active agent; and
b) at least one pharmaceutically acceptable carrier.

18. The composition of claim 17, wherein said amphiphilic block copolymer comprising at least one poly(ethylene oxide) (EO) segment and at least one poly(propylene oxide) (PO) segment.

19. The composition of claim 17, wherein said amphiphilic block copolymer has the formula: EOₓ-PO_{y}-EO_{z}, wherein x, y, and z have values from about 2 to about 300.

20. The composition of claim 17, wherein said hard tissue targeting moiety is alendronate.
